(19) United States
(12) Patent Application Publication
(10) Pub. No.: US 2006/0035827 A1
(43) Pub. Date: Feb. 16, 2006

(54) Compositions and methods for the treatment or prevention of gallbladder disease

(76) Inventor: Gary M. Green, San Antonio, TX (US)

Correspondence Address: GIFFORD, KRASS, GROH, SPRINKLE & CITKOWSKI, P.C, PO BOX 7021, TROY, MI, 48007-7021, US

(21) Appl. No.: 11/166,818

(22) Filed: Jun. 24, 2005

Related U.S. Application Data
(60) Provisional application No. 60/582,718, filed on Jun. 24, 2004.

(51) Int. Cl. A61K 38/54; A61K 31/185; A61K 31/195

(52) U.S. Cl. 514/12; 514/553; 514/562; 514/602

(57) ABSTRACT

Compositions and methods for the treatment or prevention of gallbladder disease are provided. In particular, pharmaceutical compositions containing an amount of serine proteinase inhibitor such as potato proteinase inhibitor II effective to induce gallbladder contraction in a host are provided. The compositions can be administered to individuals having a body mass index of at least 25 during rapid weight loss or during a weight loss regime.

### RELATED APPLICATION

This application claims priority of U.S. Provisional Patent Application Ser. No. 60/582,718 filed Jun. 24, 2004, which is incorporated herein by reference.

### STATEMENT REGARDING FEDERALLY SPONSORED RESEARCH OR DEVELOPMENT

Aspects of this disclosure were funded in part by Grant No. RO1-DK37482 awarded by the National Institutes of Health. Therefore, the U.S. government has certain rights in the claimed subject matter.

### BACKGROUND

1. Technical Field

The present disclosure is generally related to compositions and methods for the treatment or prevention of gallbladder disease, and, more particularly, is related to a compositions and methods using potato proteinase inhibitor II to treat or prevent gallbladder disease.

2. Related Art

Nearly two-thirds of adults in the United States are overweight, and 30.5 percent are obese, according to data from the 1999-2000 National Health and Nutrition Examination Survey (NHANES) (Flegal K. M. et al. (2002) Prevalence and trends in obesity among US adults, 1999-2000. Journal of the American Medical Association 288:1723-1727). Overweight and obesity are known risk factors for pathologies including diabetes, heart disease, stroke, hypertension, gallbladder disease, osteoarthritis (degeneration of cartilage and bone of joints), sleep apnea and other breathing problems, and some forms of cancer including uterine, breast, colorectal, kidney, and gallbladder. A recent study estimated annual medical spending due to overweight and obesity to be as much as $92.6 billion in 2002 dollars (9.1 percent of U.S. health expenditures)(Finkelstein, E. A. et al. (2003) National medical spending attributable to overweight and obesity: How much, and who's paying? Health Affairs Web Exclusive. W3:219-226. Available at http://content.healthaffairs.org/webexclusives/index.dtl?year=2003). About 3.4 billion is attributed to gallbladder disease.

Cholelithiasis, the formation of gallstones, is one type of gallbladder disease that can be readily treated. For most individuals who suffer from gallstones, the treatment of choice is to have a cholecystectomy, or surgical removal of the gallbladder. Other treatments include dissolving the gallstones with organic solvents. Additionally, the administration of ursodeoxycholic acid has been reported to prevent formation of gallstones in individuals on a weight loss regime (Broomfield, P. H., et al. (1988) Effects of ursodeoxycholic acid and aspirin on the formation of lithogenic bile and gallstones during loss of weight. N. Engl. J. Med. 319(24):1567-72).

Other treatments for gallbladder disease include administering cholecystokinin (CCK) or analogs of CCK. CCK is a polypeptide hormone which was first isolated as a 33-amino acid peptide from the porcine gastrointestinal tract. (Mutt et al. (1971) Biochem J. 125: 57-58; Mutt et al. (1976) Clin Endocrinol, supplement, 5:175-183.) Peripherally administered CCK has been shown to produce satiety in the rat, sheep and the monkey (Jorpes et al. (1964) Acta. Chem. Sianel 18:2408; Della-Fera et al. (1979) Science 206:471-73; Gibbs et al. (1973) J. Comp. Physiology and Psychology 84:488-495). Infusions of CCK-8, the synthetic C-terminal octapeptide of CCK, have been shown to decrease food intake in lean and obese men. (J. Smith (1984) Int. J. of Obesity, Vol. 8, Suppl. 1, pp. 35-38). It is now accepted that CCK has satiety-inducing effects and thus, may be useful to reduce or suppress food intake in man.

U.S. Pat. No. 5,935,948 discloses a method for treating gallstones by administering a dehydrocholic acid.

U.S. Pat. No. 5,514,088 discloses a medical device for removing gallstones from a patient.

U.S. Pat. No. 5,270,302 discloses tetrapeptide analogs which function as agonists of cholecystokinin Type-A receptor. The '302 patent also discloses the use of these compounds for the treatment or prevention of gallbladder disease.

U.S. Pat. No. 5,256,640 discloses a method for preventing gallstones using nutrient supplements containing from about 20 g to about 35 g of protein; from about 5 g to about 15 g of fat; and from about 6 g to about 15 g of carbohydrate per daily serving to contract the gallbladder.

U.S. Pat. No. 5,212,202 discloses a method for dissolving gallstones using a solvent having a C₅-C₆ ester having a boiling point in the range of 80°-40° C. in an amount and at a flow rate of at least about 4 mg/min.

U.S. Pat. No. 4,490,364 discloses analogs of CCK(1-8) useful for the contraction of the gallbladder while exhibiting either reduced or negligible gastrinic activity.

Accordingly, there is need for new compositions and methods for the treatment or prevention of gallbladder disease; in particular, there is a need for non-invasive compositions and methods for the treatment or prevention of gallbladder disease.

### SUMMARY

Aspects of the present disclosure provide methods and compositions for the treatment or prevention of gallbladder disease in a host in need of such treatment. The disclosed compositions include, but are not limited to, agents that stimulate gallbladder contraction in a host, for example by inducing endogenous secretion of CCK. Other embodiments include agents that reverse the suppression of endogenous CCK secretion, and thereby increase gallbladder motility. Endogenous CCK secretion is suppressed, for example, by pancreatic endopeptidases. Exemplary agents include, but are not limited to, proteinase inhibitors such as potato proteinase inhibitor II.

Some aspects are directed to the treatment and prevention of gallstones in a host, particularly, in a host having a body mass index of at least 25. The host can be undergoing rapid weight loss or be on a weight loss regimen such as a low calorie diet. Other aspects provide administering the disclosed proteinase inhibitors to a host on a low fat diet, or to diabetic hosts.

Additional compositions, methods, features, and advantages of the present disclosure will be or become apparent to one with skill in the art upon examination of the detailed description. It is intended that all such additional compositions, methods, features, and advantages be included within this description, be within the scope of the present disclosure, and be protected by the accompanying claims.

### DETAILED DESCRIPTION

Before the present compounds, compositions and methods are disclosed and described, it is to be understood that this disclosure is not limited to specific pharmaceutical carriers, or to particular pharmaceutical formulations or administration regimens, as such may, of course, vary. It is also to be understood that the terminology used herein is for the purpose of describing particular embodiments only and is not intended to be limiting.

1. Definitions

The term “organism” or “host” refers to any multicellular living entity having a gallbladder or the equivalent, including for example, mammals such as humans.

The term “gallbladder motility” means the processes relating to complete or partial emptying or filling of the gallbladder.

The term “biliary sludge” means material found in the gallbladder typically including, but not limited to, cholesterol monohydrate crystals, calcium bilirubinate granules, or other calcium salts. Biliary sludge may also contain a large proportion of undefined residue, protein-lipid complexes, and mucin.

The term “cholesterol saturation index” means the cholesterol mole percentage in a given bile sample divided by the maximal mole percentage of cholesterol that can be dissolved in micelles.

The term “cholesterol nucleation inhibitor” means a substance that inhibits, reduces, or interferes with the crystallization of cholesterol in the gallbladder. Exemplary cholesterol nucleation inhibitors include, but are not limited to, proteins, peptides, co-factors, surfactants, a compounds that promote the solubility of cholesterol in a biological fluid, for example bile.

The term “weight loss regimen” means a dietary plan generally including caloric restriction to reduce the weight or percentage of body fat of a host. Weight loss regimens can optionally include exercise programs. Weight loss regimens include, but are not limited to, reduced fat intake, reduced carbohydrate intake, reduced protein intake, or a combination thereof.

The term “low fat diet” means a diet in which less than 30% of total daily calories ingested or consumed are from fat.

The term “therapeutically effective amount” as used herein refers to that amount of the compound being administered which will relieve to some extent one or more of the symptoms of the disorder being treated. In reference to gallbladder disorders, a therapeutically effective amount refers to that amount which has the effect of (1) reducing or preventing the formation of gallbladder disease, (2) inhibiting (that is, slowing to some extent, preferably stopping) the formation of gallstones, (3) increasing gallbladder motility, (4) relieving to some extent (or, preferably, eliminating) one or more symptoms associated with a pathology related to or caused in part by gallstone formation, (5) preventing, reducing, or reversing biliary sludge formation or accrual; (6) treating or preventing the formation of gallbladder cancer by administration of the disclosed compounds.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “body mass index” or BMI means the ratio of an organism's weight to the organism's height squared in kg/m².

The term “overweight” means an organism having a BMI greater than about 25.

The term “obese” means an organism having a BMI of greater than about 30.

“Treating” or “treatment” of a disease includes preventing the disease from occurring in an organism that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to gallbladder disorders, these terms simply mean that the presence of gallstones is reduced or prevented in an individual or that one or more of the symptoms related to gallstones will be reduced.

The term “prodrug” refers to an agent which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, pp. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The terms “including”, “such as”, “for example” and the like are intended to refer to exemplary embodiments and not to limit the scope of the present disclosure.

The term “proteinase” is used interchangeably with “peptidase” and refers to enzymes that cleave peptide bonds.

The term “peptidase” refers to two groups of enzymes: the endopeptidases which cleave peptide bonds at points within the protein, and the exopeptidases which remove amino acids sequentially from either the N or C-terminus.

The term “proteinase” is also used as a synonym word for endopeptidase and four mechanistic classes of proteinases. The four classes are the serine proteinases, cysteine proteinases, aspartic proteinases, and the metallo proteinases.

The term “proteinase inhibitor” refers to an enzyme, variant, fragment, or prodrug of the enzyme that is capable of reducing the rate of peptide bond cleavage by a proteinase; and is characterized by at least fifteen amino acid bases and one binding site for trypsin or chymotrypsin or elastase.

The term “lithogenic” means promoting the formation of calculi or mineral salts.

The term “rapid weight loss” means losing at least about three pounds a week.

The term “low calorie diet” generally refers to a diet of less than the U.S. recommended daily caloric intake. The U.S. recommended daily allowance is 2300-3000 calories for men and 1900-2200 calories for women.

All stereoisomers of the present compositions, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The chiral centers of the compounds of the present disclosure can have the S or R configuration as defined by the IUPAC 1974 Recommendations.

2. Proteinase Inhibitors

Some embodiments of the present disclosure include compositions such as pharmaceutical compositions having an amount of an agent effective to reduce or prevent gallbladder disease in a host. One embodiment provides a composition having an agent that reduces or prevents the formation of gallstones in a host. Exemplary agents include proteinase inhibitors such as proteinase inhibitors, for example, serine proteinase inhibitors that inhibit trypsin, chymotrypsin, elastase or a combination thereof.

The serine proteinases generally include two distinct families: (1) the chymotrypsin family which includes the mammalian enzymes such as chymotrypsin, trypsin or elastase or kallikrein, and (2) the substilisin family which include the bacterial enzymes such as subtilisin. The serine proteinases exhibit different substrate specificities which are related to amino acid substitutions in the various enzyme subsites interacting with the substrate residues. Some enzymes have an extended interaction site with the substrate whereas others have a specificity restricted to the P1 substrate residue. It will be appreciated by one of skill in the art that any serine proteinase inhibitor may be used provided the serine proteinase inhibitor directly or indirectly causes an increase in levels of endogenous CCK in amounts sufficient to increase gallbladder motility, for example, in a host under a diet regimen such as a low fat regimen.

During the enzymatic process, three residues form the catalytic triad essential in the catalytic process i.e., His 57, Asp 102 and Ser 195 (chymotrypsinogen numbering). The first step in the catalysis is the formation of an acyl enzyme intermediate between the substrate and the essential Serine. Formation of this covalent intermediate proceeds through a negatively charged tetrahedral transition state intermediate and then the peptide bond is cleaved. During the second step or deacylation, the acyl-enzyme intermediate is hydrolyzed by a water molecule to release the peptide and to restore the Ser-hydroxyl of the enzyme. The deacylation, which also involves the formation of a tetrahedral transition state intermediate, proceeds through the reverse reaction pathway of acylation. A water molecule is the attacking nucleophile instead of the Ser residue. The His residue provides a general base and accept the OH group of the reactive Ser.

Suitable proteinase inhibitors include, but are not limited to, trypsin inhibitors or pancreatic proteinase inhibitors. Representative trypsin inhibitors include members of the Kunitz-type inhibitor family and have a molecular mass of about 20-25 kDa. Kunitz-type inhibitors are known in the art and are specific for trypsin, and to a lesser extent chymotrypsin. Proteins from the Kunitz family contain from 170 to 200 amino acid residues and one or two intra-chain disulphide bonds with a conserved region in their N-terminal section.

Additional representative serine proteinase inhibitors include Bowman-Birk proteinase inhibitors. The Bowman-Birk inhibitor family is one of the numerous families of serine proteinase inhibitors (Laskowski M., Kato I. (1980) Protein inhibitors of proteinases. Annu. Rev. Biochem. 49:593-626). Bowman-Birk proteinase inhibitors are about 6-10 kD and inhibit trypsin, chymotrypsin, and elastase. They have a duplicated structure and generally possess two distinct inhibitory sites. These inhibitors are primarily found in plants and in particular in the seeds of legumes as well as in cereal grains.

Proteins of the Bowman-Birk inhibitor family of serine proteinase inhibitors interact with the enzymes they inhibit via an exposed surface loop that adopts the canonical proteinase inhibitory conformation. The resulting non-covalent complex renders the proteinase inactive. This inhibition mechanism is common for the majority of serine proteinase inhibitor proteins and many analogous examples are known. A particular feature of the Bowman-Birk inhibitor protein, however, is that the interacting loop is a particularly well-defined disulfide-linked short beta-sheet region.

The proteinase inhibitor can be a single proteinase inhibitor, an inhibitor of more than one proteinase, or at least two proteinase inhibitors can be combined. For example, a trypsin inhibitor can be combined with a chymotrypsin inhibitor. Alternatively, a single inhibitor effective against both trypsin and chymotrypsin can be used. The proteinase inhibitors can be reversible or irreversible inhibitors. Representative proteinase inhibitors include, but are not limited to, aprotinin; potato proteinase inhibitor II, antithrombin III; 4-amidino-phenyl-methane sulfonyl fluoride (APMSF); chymostatin; phenylmethylsulfonyl fluoride; N-tosyl-L-phenylalanine chloromethyl ketone (TLCK); 1-chloro-3-tosylamido-7-amino-L-2-heptnanone; Na-p-tosyl-L-lysine chloromethyl ketone hydrochloride; 1-chloro-3-(4-tosyl-amido)-4-phenyl-2-butanone (TPCK); or a combination thereof.

A pharmaceutical composition is provided herein containing an amount of potato proteinase inhibitor II effective to induce gallbladder contraction in an individual undergoing a weight loss regimen. The potato proteinase inhibitor II can be substantially free of other proteinase inhibitors including, but not limited to, Kunitz, Bowman-Birk, carboxypeptidase inhibitors, or a combination thereof. Substantially free of other proteinase inhibitors means at least about 90% by weight potato proteinase inhibitor II, typically at least about 95% potato proteinase inhibitor II, more typically at least about 98% potato proteinase inhibitor II by weight. Typically, the host is a human having a BMI of at least about 25, more typically, at least about 30, and optionally is undergoing a low fat or low calorie diet regimen.

The active ingredient in an inventive composition can be an agent, for example a non-caloric agent, that induces the secretion or release of endogenous CCK-58 in a host. CCK-58 is an endogenous form of cholecystokinin that has a unique spectrum of actions. Unlike CCK-8, CCK-58 does not induce pancreatitis when administered in large supramaximal doses.

Alternatively, the active ingredient of the disclosed compositions is a pancreatic endopeptidase inhibitor. Inhibition of pancreatic endopeptidases blocks the suppressive effect of pancreatic endopeptidases on CCK secretion.

2.1 Potato Proteinase Inhibitor II

Proteinase inhibitor II obtained from potato is an inhibitor of chymotrypsin and trypsin and is a heat stable protein, and pepsin and acid stable. It has a dimeric molecular weight of 21,000. Four monomeric isoinhibitor species of molecular weight 10,500 comprise inhibitor II and have been isolated by chromatography in the presence of urea. Upon removal of the urea, each monomeric species dimerized to yield homogenous dimers. The three major protomer species, called B, C, and D were found to have similar molecular weights and amino acid compositions, and each has an N-terminal alanine residue. Reconstituted dimers possess two binding sites for bovine α-chymotrypsin, indicating that each monomer possesses one binding site for this enzyme. Significant differences have been noted among the reconstituted dimers in their isoelectric points, immunoelectrophoretic mobilities, ion-exchange properties, and their inhibitory reactivities against trypsin. The properties of the inhibitor II dimeric species are similar but not identical to inhibitors IIa and IIb reported from Japanese potatoes, indicating the existence of intervarietal, as well as intravarietal, differences among potato tuber inhibitor II isoinhibitors (Bryant, J., et al. (1976) Proteinase inhibitor II from potatoes: Isolation and characterization of its protomer components, Biochemistry 15:3418-3424).

Proteinase inhibitor II is composed of two sequence repeats. It contains two reactive site domains. The role of the two reactive sites in the inhibition of trypsin and chymotrypsin has been evaluated. The first reactive site inhibits only chymotrypsin (Ki=2 nM), and this activity is very sensitive to mutations. The second reactive site strongly inhibits trypsin (Ki=0.4 nM) and chymotrypsin (Ki=0.9 nM), and is quite stable towards mutations (Beekwilder, J. et al. (2000) Characterization of potato proteinase inhibitor II reactive site mutants. Eur. J. Biochem. 267:1975-1984). Potato proteinase II has been shown to be active in humans and to stimulate CCK secretion (Schwartz, J. G., et al. (1994) Treatment with an oral proteinase inhibitor slows gastric emptying and acutely reduces glucose and insulin levels after a liquid meal in type II diabetic patients. Diabetes Care. 17(4):255-62; Spreadbury, D. et al. (2003) A proteinase inhibitor extract from potatoes reduces post-prandial blood glucose in human subjects. JANA 6:29-38). Potato Proteinase inhibitor. II is commercially available under the trade name SATISE™ (Kemin Industries, Des Moines, Iowa).

Variations of potato proteinase inhibitor II are within the scope of this disclosure. A variation or variant refers to a polypeptide that differs from potato proteinase inhibitor II or other agent that stimulates endogenous secretion of CCK, but retains the ability to stimulate endogenous secretion of CCK or induce gallbladder contraction. A typical variant differs in amino acid sequence from potato proteinase inhibitor II. The sequence of potato proteinase inhibitor II is known in the art (Murray, C. Christeller, J. T. (1994) Genomic Nucleotide Sequence of a Proteinase Inhibitor II Gene. Plant Physiol. 106:1681; EMBL accession number X78275, both of which are incorporated by reference herein in their entirety). Generally, differences are limited so that the sequences of the proteinase inhibitor and the variant are closely similar overall and, in many regions, identical. A variant and proteinase inhibitor may differ in amino acid sequence by one or more modifications (e.g., substitutions, additions, and/or deletions). A substituted or inserted amino acid residue may or may not be one encoded by the genetic code. A variant of a proteinase inhibitor may be naturally occurring such as an allelic variant, or it may be a variant that is not known to occur naturally.

Modifications and changes can be made in the structure of the proteinase inhibitors of the disclosure and still obtain a molecule having similar characteristics as the proteinase inhibitor (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

A proteinase inhibitor fragment according to the present invention has at least fifteen amino acid bases and at least one binding site for either trypsin or chymotrypsin. With reference to potato proteinase inhibitor II, an operative fragment of the 154 amino acid polypeptide domain or variant thereof includes either a first domain fragment made up of the reactive site loop encoded in the first 27 amino acids along with amino acid substitutions or additional polypeptides to mimic the first domain of potato proteinase inhibitor II (Greenblatt, H. M. et al (1989) Structure of the complex of Streptomyces griseus proteinase B and polypeptide chyotrypsin inhibitor-1 from Russet Burbank potato tubers at 2.1 Å resolution. J. Mol. Biol. 205, 201-208). An alternate potato proteinase inhibitor II fragment includes a second reactive domain that is inhibitory of both trypsin and chymotrypsin. The second reactive domain fragment includes amino acid residues 61-64 with a complete protein (NCBI CAA55082). Preferably, the second reactive domain fragment encompasses amino acid residues 58-74. More preferably, a second domain fragment includes reactive moieties such as cysteine residue substitutions so as to mimic the second reactive site loop associated with a native potato proteinase inhibitor II.

Potato cysteine proteinase inhibitor is a 180 amino acid protein (NCBI S38742), is a 22 kDa fragment formed by proteolysis of 85 kDa crystalline cysteine proteinase inhibitor (Walsh, T. A. and Strickland, J. A. (1993) Proteolysis of the 85-kilodalton crystalline cysteine proteinase inhibitor from potato releases function cystatin domains. Plant Physiology 103:1227-1234). This fragment as well as the 10 kDa fragments formed by cleavage of the 85 kDa inhibitor polypeptide, are all appreciated to be operative to bind trypsin and/or chymotrypsin.

2.2 Source of Proteinase Inhibitors

The proteinase inhibitors described herein can be obtained from commercial sources or natural sources including plants such as potatoes or tomatoes (Nelson, C. E. and Ryan, C. A. (1980) Temporal shifts in the apparent in vivo translational efficiencies of tomato leaf proteinase inhibitors I and II mRNAs following wounding. Biochem Biophys Res Commun. 94(1):355-9), and the like. Methods for purifying potato proteinase inhibitor II are known in the art. See for example, U.S. Pat. No. 6,686,456 which is incorporated by reference in its entirety. In particular, potato proteinase inhibitor II has been isolated and purified (Bryant, et al. (1976) Proteinase Inhibitor II from Potatoes: Isolation and Characterization of its Protomer Components Biochemistry 15(16): 3418-3423). The sequence of various proteinase inhibitors is also known in the art (see U.S. Pat. No. 6,440,727 and GenBank). Methods for obtaining the sequence of purified proteins are also known in the art.

2.2.1 Recombinant Polypeptides

Recombinant proteinase peptides or recombinant proteinase inhibitor peptides can also be used in the present disclosure. The recombinant proteinase inhibitors can be modified to resist degradation, for example degradation by digestive enzymes and conditions. Techniques for the expression and purification of recombinant proteins are known in the art (see Sambrook Eds., Molecular Cloning: A Laboratory Manual 3^{rd} ed. (Cold Spring Harbor, N.Y. 2001).

Some embodiments of the present disclosure provided compositions containing proteinase inhibitors that can be expressed as encoded polypeptides or proteins. The engineering of DNA segment(s) for expression in a prokaryotic or eukaryotic system may be performed by techniques generally known to those of skill in recombinant expression. It is believed that virtually any expression system may be employed in the expression of the claimed nucleic and amino sequences.

Generally speaking, it may be more convenient to employ as the recombinant polynucleotide a cDNA version of the polynucleotide. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the inventor does not exclude the possibility of employing a genomic version of a particular gene where desired.

As used herein, the terms “engineered” and “recombinant” cells are intended to refer to a cell into which an exogenous DNA segment or gene, such as a cDNA or gene has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic DNA, and also include genes positioned adjacent to a promoter not naturally associated with the particular introduced gene.

To express a recombinant encoded polypeptide proteinase or proteinase inhibitor in accordance with the present disclosure one would prepare an expression vector that comprises a polynucleotide under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli .chi. 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda may be utilized in making a recombinant phage vector that can be used to transform host cells, such as E. coli LE392.

Further useful vectors include pIN vectors and pGEX vectors, for use in generating glutathione S-transferase (GST) soluble fusion proteins for later purification and separation or cleavage. Other suitable fusion proteins are those with β-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to microorganisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autographica californica nuclear polyhedrosis virus (AcNPV) is used as a vector to express foreign genes. The virus grows in Spodoptera frugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

It is contemplated that the isolated nucleic acids of the disclosure may be “overexpressed”, i.e., expressed in increased levels relative to its natural expression in human cells, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or immunoblotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

2.2.1.1 Purification of Expressed Proteins

Further aspects of the present disclosure concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state, i.e., in this case, relative to its purity within a hepatocyte or p-cell extract. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification are well known to those of skill in the art. These include, for example, precipitation with ammonium sulphate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

3. Gallbladder Disease

The disclosed compositions and methods can be used in the treatment or prevention of gallbladder disease, for example gallstones, decreased gallbladder motility, gallbladder stasis, and gallbladder cancer. Certain embodiments are directed to treating or preventing gallbladder disease in overweight or obese individuals, in particular individuals having a BMI of at least about 25, typically at least about 30. The compositions and methods can be used during weight loss regimens or during rapid weight loss, for example following bariatric surgery or during a diet regimen. Rapid weight loss generally refers to loss of at least about three pounds in a week. The U.S. recommended daily allowance is 2300-3000 calories for men and 1900-2200 calories for women. Accordingly, a diet regimen refers to limiting caloric intake to less than the U.S. recommended daily allowance. It will be appreciated by those of skill in the art that the daily calorie intake to maintain a current weight can vary between individuals and can depend on the activity level of the individual.

3.1 Gallstones

As noted above, cholelithiasis is a significant medical concern. In particular, overweight and obese individuals are known to be at risk for developing gallstones for example during rapid weight loss or during low calorie, low fat diet regimens (Festi, D. et al. (2000) Review: low caloric intake and gall-bladder motor function. Aliment Pharmacol Ther. Suppl 2:51-3; Liddle, R. A. et al. (1989) Gallstone formation during weight-reduction dieting. Arch Intern Med. 149(8):1750-3). Women with a BMI of at least about 30 are reported to have at least double the risk for gallstone formation as women with a BMI of less than about 25 (Everhart, J. (1993) Contributions of obesity and weight loss to gallstone disease. Annals Internal Med. 119(10):1029-35).

Some embodiments of the present disclosure can be used to treat or prevent cholesterol or pigmented gallstones. Cholesterol stones are hard, crystalline stones that contain more than 50% cholesterol plus varying amounts of protein and calcium salts. Pigment stones consist of several insoluble calcium salts that are not normal constituents of bile.

Without wishing to be bound by any one theory, it is believed that at least three physical conditions are necessary for the formation of cholesterol gallstones. First, the bile must become supersaturated with cholesterol. The liver, perhaps as a result of genetic programming, produces supersaturated bile by a decreased secretion of bile salts, an increased secretion of cholesterol, or both. Reduced bile flux through the liver produces lithogenic bile with excess cholesterol. Obesity is associated with excess cholesterol production. The cholesterol saturation index, based on the relative concentrations of cholesterol, bile salts, and phospholipids, is a commonly used indicator of the presence of a saturation defect. A cholesterol saturation index greater than 1 is considered supersaturated.

The second physical condition is the presence of a kinetic defect that accelerates cholesterol crystal nucleation and growth in supersaturated bile. Retention occurs in the gallbladder because the epithelium secretes excess mucus (consisting of mucin, a glycoprotein). This mucus gel forms a colloidal shell that entraps cholesterol microcrystals, preventing them from being ejected from the gallbladder. Mucin also creates a scaffold for the addition of more crystals. A defect in the contractile function of the gallbladder smooth muscle results in failure to properly evacuate the solid material. Several antinucleating and pronucleating proteins, perhaps derivatives of gallbladder mucin, have been implicated as kinetic factors. Cholesterol crystal formation (visible by microscopy) followed by sludge formation (visible by gallbladder ultrasonography) are thought to be necessary precursors to cholesterol gallstone formation. “Biliary sludge” consists of calcium bilirubinate, cholesterol microcrystals and mucin.

The third requirement for gallstone formation is gallbladder hypomotility, which produces stasis of bile in the gallbladder, increased cholesterol supersaturation, and opportunities for cholesterol crystallization as well as gallstone formation and growth. In vivo, gallbladder hypomotility is measured by increased fasting volume, increased contracted volume, and decreased ejection fraction. Perhaps two of these three physical conditions, a kinetic defect and gallbladder hypomotility, may also predispose persons to pigment gallstone formation.

In North America, black pigment stones constitute about 15% of gallstones found at surgery (cholecystectomy). They are frequently associated with hemolysis or alcoholic cirrhosis. The basis for their formation is excessive bilirubin excretion in bile. Brown pigment stones are associated with stagnation and infection (often from a stricture) or infestation (e.g., liver flukes) of the biliary tract. Such conditions predispose to chronic cholangitis and eventually cholangiocarcinoma. Infection and inflammation increase b-glucuronidase, an enzyme that deconjugates bilirubin; the resultant free bilirubin then polymerizes and complexes with calcium, forming calcium bilirubinate in the bile duct system.

4. Methods of Use

A method of treating or preventing gallbladder disease in a host is provided herein by administering to the host an amount of an agent that stimulates the endogenous secretion of CCK or reduces inhibition of endogenous secretion of CCK in a host sufficient to induce contraction of the host's gallbladder. A representative agent includes, but is not limited to proteinase inhibitors, typically serine proteinase inhibitors, more typically potato proteinase inhibitor II. A host undergoing rapid weight loss or on a low calorie diet is an indication for an inventive treatment.

According to the present invention, a host is identified that has a risk of developing gallbladder disease by a measurement of CCK levels in an individual host. Extrapolation of biolevels of CCK from sera levels is common to the art. Through the administration of an inventive composition, CCK levels are increased within the host in an amount sufficient to increase gallbladder motility but by affording less time for stone development to occur. Thereafter, the host is monitored for CCK higher relative levels associated with increased gallbladder motility. The increased gallbladder motility is believed to afford less time for gallbladder sludge to form and nucleate gallstones.

Low calories diets include diets of about 2000 calories per day or less, typically about 1500 calories per day or less, even more typically about 1000 calories per day or less. The diet regimen can also be a low fat diet regimen, for example where the calories from fat are about 30% or less of total daily calories.

A host having a body mass index of at least about 30, and typically of at least about 25, can have a decreased sensitivity to CCK compared to the general population, thus, requiring more CCK to be secreted to induce gallbladder contraction in the host. Such decreased sensitivity can be genetic or the result of a prolonged high fat diet or obesity. Alternatively, the host secretes low levels of CCK in response to food intake. Such low level secretion of CCK may not be enough to properly contract the gallbladder and may lead to the formation of gallstones and therefore is also an indication for an inventive treatment. Low CCK levels are detected through standard blood screening.

Low levels of endogenously secreted CCK can be increased by administering the disclosed compositions. For example, pancreatic proteinase activity reduces the secretion of CCK, through the administration of a pancreatic proteinase inhibitor, and in particular a pancreatic endopeptidase inhibitor, to a host reduces inhibition of CCK by endogenous pancreatic endopeptidases. Additionally, the host can be diabetic.

A method for treating or preventing gallbladder disease in a host having a cholesterol saturation index of bile of at least 1 is provided by administering the disclosed compositions to the host. Generally, the disclosed compositions induce gallbladder emptying and remove the supersaturated bile from the gallbladder prior to the formation of gallstones or cholesterol crystals, i.e., lithogenic bile.

A method for preventing gallstone formation in a host is also provided by administering to the host a pharmaceutical composition including potato proteinase inhibitor II, a variant, fragment, prodrug, or pharmaceutically acceptable salt thereof in an amount sufficient to stimulate gallbladder emptying to prevent or reduce the formation of gallstones. In particular, the compositions and methods are effective on a host that has undergone bariatic surgery including, but not limited to, gastric bypass or a gastric restriction procedure. Bariatric surgery includes but is not limited to Vertical Banded Gastroplasty (VBG), Laparoscopic Adjustable Band and Roux En-Y Gastroplasty.

A method for treating or preventing gallbladder disease in host during a weight loss regimen is provided by administering to the host a pharmaceutical composition including an endogenous CCK-58 releasing agent in an amount sufficient to stimulate gallbladder emptying during a weight loss regimen and reduce or prevent the formation of gallstones. Endogenous CCK-58 releasing agents include serine proteinase inhibitors such as potato proteinase inhibitor II and induce the secretion of CCK-58 by the host.

A method for treating or preventing gallbladder disease in host, for example during a weight loss regimen is provided by administering to the host a pharmaceutical composition including a pancreatic endopeptidase inhibitor in amount sufficient to reduce suppression of endogenous CCK-58 secretion by pancreatic endopeptidases and thereby stimulate gallbladder emptying during a weight loss regimen and reduce or prevent the formation of gallstones.

Improved gallbladder motility associated with the present invention is illustratively identified by performing a gallbladder ultrasound scans in order to detect gallbladder size, before and after contraction. Preferably, a gallbladder baseline motility scan is performed prior to initiating a treatment according to the present invention. Alternatively, or in conjunction with ultrasound scans, blood serum levels of CCK are measured by conventional tests.

The compositions containing potato proteinase inhibitor II are optionally taken orally with a meal, preferably with a meal that is part of a low calorie diet regimen. Typically, the compositions are taken concurrently with eating a meal or prior to eating a meal. In some embodiments, the disclosed compositions can be taken once daily, twice daily, or as needed with each meal, snack, or other consumption of foodstuffs.

5. Combination Therapy

The disclosed compositions can include or be used in combination with one or more additional therapeutic agents. Representative additional therapeutic agents include, but are not limited to, a non-steroidal anti-inflammatory compound, an HMG-CoA reductase inhibitor, a cholesterol nucleation inhibitor, dietary fiber, or combinations thereof.

Exemplary HMG-CoA reductase inhibitors include, but are not limited to, atorvastatin, cerivastatin, fluvastatin, lovastatin, pravastatin, rosuvastatin, and simvastatin. Such inhibitors are known to reduce cholesterol concentrations.

Representative non-steroidal anti-inflammatory compounds include, but are not limited to, aspirin, diclofenac, piroxicam, indomethacin, acetaminophen, ibuprofen, naproxen, ketoprofen, and cox-2 inhibitors, and combinations thereof. Cox-2 inhibitors include celecoxib and rofecoxib and combinations thereof. Such anti-inflammatories are administered in a regimen to reduce gallbladder inflammation typical of gallstone formation conditions, thereby facilitating motility.

Exemplary cholesterol nucleation inhibitors include apolipoproteins such as A-I or A-II. These nucleation inhibitors decrease the kinetics of lithogenic cholesterol formation.

An inventive composition in combination with a bile-acid-binding resin is also operative herein. Bile-acid-binding resins, in particular, cholestryamine, stimulate CCK release by reversing the suppressive effects of bile acids on CCK release.

Dietary fiber is recognized to increase CCK levels as well as reduce cholesterol levels. Dietary fiber is readily ingested with a proteinase to promote gallbladder health.

Combination therapy is attractive because the combination is operative to lower the dose of each ingredient required to stimulated gallbladder contraction since multiple modes of gallbladder stone forming disfunction are simultaneously treated; the modes inclusive of high cholesterol concentration, cholesterol crystallization, gallbladder motility and gallbladder inflammation. This is especially so if the compounds interact synergistically, i.e., if they potentiate each other. Also combinations of agents may have a greater maximal effect than the individual agents given separately.

The amount of cholestyramine required to strongly stimulate gallbladder contraction is about 4 grams, which will bind about one pool of bile acids (approximately 2.5-3.5 g) in humans. Administration of a proteinase inhibitor in combination with 4 grams or less of cholestyramine in a dose serves to promote normal gallbladder function.

6. Dosage

Determination of the appropriate dose of the proteinase inhibitor to induce gallbladder contraction can be determined in a dose-response study in a statistically significant cohost, preferably of about thirty female subjects without gallbladder disease. More preferably, the subjects are at risk of developing gallbladder disease. The subjects can be screened for gallbladder disease using ultrasound. Blood CCK levels can be monitored under three conditions: (1) in response to a typical fat-containing meal, (2) in response to a low fat meal without potato proteinase inhibitor II, and (3) in response to a low fat meal taken with potato proteinase inhibitor II. Gallbladder emptying can also be monitored by gamma scintigraphy using the gallbladder imaging agent, ^{99m}Tc-mebrofenin (8 mCi injected 1 hour prior to administration of the meal). Stomach emptying can be monitored simultaneously with gallbladder imaging using ^{99m}Tc-sulfur colloid (1 mCi administered with the meal) to confirm the effect of potato proteinase inhibitor 2 on this parameter.

Similar studies are readily performed on obese subjects. The obese subjects can be screened with ultrasound prior to dieting and those without gallbladder disease can be selected. The subjects can be divided into two groups, a low calorie diet group treated with potato proteinase inhibitor II (Group 1) and a low calorie diet without potato proteinase inhibitor II (Group 2). The groups can be monitored for about 8 weeks during dieting. Ultrasound scanning for indications of gallbladder disease, i.e., sludge and gallstone formation, can be repeated at 4 weeks and 8 weeks. Blood CCK levels in response to the test meals with and without potato proteinase inhibitor II can also be determined at 4 weeks and at 8 weeks. A fourth ultrasound study can be performed at 6 months after completion of the study to assess long term outcome of diet and therapy.

Dose-response curves relating potato proteinase inhibitor II with gallbladder emptying and blood CCK levels can be established to mimic gallbladder emptying caused by normal meals.

7. Pharmaceutical Compositions

Pharmaceutical compositions and dosage forms include a pharmaceutically acceptable salt of disclosed or a pharmaceutically acceptable polymorph, solvate, hydrate, dehydrate, co-crystal, anhydrous, or amorphous form thereof. Specific salts of disclosed compounds include, but are not limited to, sodium, lithium, potassium salts, and hydrates thereof.

Pharmaceutical compositions and unit dosage forms of the disclosure typically also include one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a dosage form used in the acute treatment of a disease or disorder may contain larger amounts of the active ingredient, for example the disclosed compounds or combinations thereof, than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 20^{th} ed.

Typical pharmaceutical compositions and dosage forms include one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure comprise a pharmaceutically acceptable salt, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, more preferably in an amount of from 50 mg to 500 mg, even more preferably in an amount of from about 30 mg to about 100 mg.

Additionally, the compounds and/or compositions can be delivered using lipid- or polymer-based nanoparticles. For example, the nanoparticles can be designed to improve the pharmacological and therapeutic properties of drugs administered parenterally (Allen, T. M., Cullis, P. R. Drug delivery systems: entering the mainstream. Science. 303(5665): 1818-22 (2004)).

7.1. Oral Dosage Forms

Pharmaceutical compositions of the disclosure that are suitable for oral administration can be presented as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 20^{th} ed.

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 total weight percent of the pharmaceutical compositions or dosage forms.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients which are well known in the art and are listed in the USP (XXI)/NF (XVI), which is incorporated herein by reference. In general, lactose-free compositions comprise a pharmaceutically acceptable salt of a proteinase inhibitor, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms including the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2^{nd} ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

7.2 Controlled and Delayed Release Dosage Forms

Pharmaceutically acceptable salts of the disclosed compounds can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420.

Conventional OROS® oral dosage forms are made by compressing a drug powder (e.g., a proteinase inhibitor salt) into a hard tablet, coating the tablet with cellulose derivatives to form a semi-permeable membrane, and then drilling an orifice in the coating (e.g., with a laser). Kim, Cherng-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium.

A specific dosage form of the compositions of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer comprises a salt of an proteinase inhibitor, or a polymorph, solvate, hydrate, dehydrate, co-crystal, anhydrous, or amorphous form thereof. See U.S. Pat. No. 6,368,626.

Another specific dosage form of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of a proteinase inhibitor, or a polymorph, solvate, hydrate, dehydrate, co-crystal, anhydrous, or amorphous form thereof. See U.S. Pat. No. 6,342,249.

7.3. Parenteral Dosage Forms

Parenteral dosage forms can be administered to patients by various routes, including, but not limited to, subcutaneous, intravenous (including bolus injection), intramuscular, and intra-arterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a proteinase inhibitor disclosed herein can also be incorporated into the parenteral dosage forms of the disclosure, including conventional and controlled-release parenteral dosage forms.

7.4. Topical Transdermal and Mucosal Dosage Forms

Topical dosage forms of the disclosure include, but are not limited to, creams, lotions, ointments, gels, shampoos, sprays, aerosols, solutions, emulsions, and other forms know to one of skill in the art. See, e.g., Remington's Pharmaceutical Sciences, 20^{th} ed.; and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 20^{th} ed.

Transdermal and mucosal dosage forms of the compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 20^{th} ed.; and Introduction to Pharmaceutical Dosage Forms, 4^{th} Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of an proteinase inhibitor of the disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of an proteinase inhibitor can be used to further adjust the properties of the resulting composition.

8. Kits

Typically, active ingredients of the pharmaceutical compositions of the disclosure are preferably not administered to a patient at the same time or by the same route of administration. This disclosure therefore encompasses kits which, when used, for example by the medical practitioner, can simplify the administration of appropriate amounts of active ingredients to a patient.

A typical kit includes a unit dosage form of a pharmaceutically acceptable salt of a proteinase inhibitor and optionally, a unit dosage form of a second pharmacologically active compound, such as anti-proliferative agent, or anti-cancer agent. In particular, the pharmaceutically acceptable salt of an proteinase inhibitor is the sodium, lithium, or potassium salt, or a polymorph, solvate, hydrate, dehydrate, co-crystal, anhydrous, or amorphous form thereof. A kit may further include a device that can be used to administer the active ingredient. Examples of such devices include, but are not limited to, syringes, drip bags, patches, and inhalers.

Kits of the disclosure can further comprise pharmaceutically acceptable vehicles that can be used to administer one or more active ingredients (e.g., a proteinase inhibitor). For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

1. A combination therapy composition comprising:
a therapeutic amount of a proteinase inhibitor effective to increase serum levels of cholecystokinin; and
a non-proteinase therapeutic agent operative to modify a gallbladder function selected from the group consisting of: decreasing cholesterol concentration in bile, decreasing cholesterol crystallization rate, decreasing gallbladder inflammation and increasing gallbladder motility.

2. The composition of claim 1 wherein the cholecystokinin is CCK-58.

3. The composition of claim 1 wherein said proteinase inhibitor comprises potato proteinase inhibitor II.

4. The composition of claim 1 wherein said proteinase inhibitor comprises a pancreatic endopeptidase inhibitor.

5. The composition of claim 1 wherein said proteinase inhibitor is selected from the group consisting of: aprotinin; antithrombin III; APMSF; chymostatin; phenylmethylsulfonyl fluoride; TLCK; 1-chloro-3-tosylamido-7-amino-L-2-heptnanone; Na-p-tosyl-L-lysine chloromethyl ketone hydrochloride; TPCK; 1-chloro-3-(4-tosyl-amido)-4-phenyl-2-butanone; N-tosyl-L-phenylalanine chloromethyl ketone; or a combination thereof.

6. The composition of claim 1 wherein said therapeutic agent is selected from the group consisting of: a nonsteroidal anti-inflammatory compound, an HMG-CoA reductase inhibitor, a cholesterol nucleation inhibitor, dietary fiber and combinations thereof.

7. The composition of claim 2 wherein said non-proteinase inhibitor therapeutic agent is an HMG-CoA reductase inhibitor.

8. The composition of claim 2 wherein said non-proteinase inhibitor therapeutic agent is dietary fiber.

9. The composition of claim 1 wherein said non-proteinase inhibitor therapeutic agent is a bile acid binding resin cholestyramine.

10. The composition of claim 6 wherein said non-proteinase inhibitor therapeutic agent is said cholesterol nucleation inhibitor, said cholesterol nucleation inhibitor being apolipoprotein.

11. The composition of claim 1 wherein said proteinase inhibitor and said non-proteinase inhibitor therapeutic agent are combined in a single unit dosage form.

12. A method of treating or preventing gallbladder disease in a host comprising:
identifying a host having a cholecystokinin blood serum level;
administering to said host an amount of a proteinase inhibitor sufficient to increase cholecystokinin in the blood serum; and
monitoring said host for gallbladder motility subsequent to administration.

13. The method of claim 12 wherein said host is commencing a weight loss regimen.

14. The method of claim 12 wherein said host has a body mass index of at least 25.

15. The method of claim 12 wherein said proteinase inhibitor is potato proteinase inhibitor II.

16. The method of claim 12 further comprising administration of a non-proteinase inhibitor therapeutic agent selected from the group consisting of: a nonsteroidal anti-inflammatory compound, an HMG-CoA reductase inhibitor, a cholesterol nucleation inhibitor, dietary fiber and combinations thereof.

17. The process of claim 12 further comprising repeating the administration step at regular intervals while said host is involved in a weight loss regimen and weighing said host at regular intervals.

18. The method of claim 12 wherein administration to said host is oral.

19. A commercial kit comprising: a unit dosage form of a pharmaceutically acceptable salt of a proteinase inhibitor and instructions for the use thereof for administration to a host so as to increase gallbladder motility.

20. The kit of claim 19 further comprising a unit dosage form of a second pharmacologically active compound selected from the group consisting of: an antiproliferative agent and an anticancer agent.

21. The kit of claim 19 further comprising a device suitable for administration of said pharmaceutically suitable salt of said proteinase inhibitor.
